(19) United States
(12) Patent Application Publication
(10) Pub. No.: US 2022/0076798 A1
(43) Pub. Date: Mar. 10, 2022

(54) CONSUMPTION APPARATUS

(71) Applicant: Radient Technologies Innovations Inc., Edmonton (CA)

(72) Inventors: Michael Cabigon, Edmonton (CA); Jim Seethram, Edmonton (CA); Steven Splinter, Edmonton (CA); Denis Taschuk, Edmonton (CA)

(21) Appl. No.: 17/240,651

(22) Filed: Apr. 26, 2021

Related U.S. Application Data
(63) Continuation of application No. PCT/IB2019/058787, filed on Oct. 15, 2019.
(60) Provisional application No. 62/750,212, filed on Oct. 24, 2018.

(51) Int. Cl. G16H 20/13; A24F 13/00; A24F 40/53; A24F 47/00; A61M 11/04

(52) U.S. Cl. CPC G16H 20/13; A24F 13/00; A24F 40/53; A24F 47/00; A61M 11/041; A61M 2205/3368; A61M 2205/502; A61M 2205/3553; A61M 2205/75; A61M 2205/3306; A61M 2205/50

(57) ABSTRACT

Systems and methods are provided for the discreet consumption of cannabis. The system includes a hot box that includes one or more cannabis provision devices to provide cannabis products to a user. The hot box further includes a cannabis safety module, one or more amenities devices, a hot box operations device, and an additional operations device. The hot box is configured to communicate with one or more user devices.

### CROSS-REFERENCE TO RELATED APPLICATIONS

The present patent application is a continuation of International Application No. PCT/IB2019/058787 filed Oct. 15, 2019, which claims the priority benefit of U.S. provisional patent No. 62/750,212 filed Oct. 24, 2018, the disclosures of which are incorporated by reference herein.

### BACKGROUND OF THE INVENTION

### 1. Field of Disclosure

The present disclosure is generally related to the consumption of cannabis products and specifically to the discreet consumption of cannabis in public or pseudo-public places.

### 2. Description of the Related Art

In many jurisdictions, cannabis can be used for medical and adult recreational purposes. Various forms and delivery methods are available, including for example, dried flower and vaping liquids for inhalation, and extracts and concentrates for oral, nasal and transdermal administration. Some users may wish for their consumption of cannabis to remain discreet while in public or pseudo-public places.

### SUMMARY OF THE INVENTION

Embodiments of the present invention provide a system and method for discreet consumption of cannabis. A standalone unit (a “hot box”) is utilized for users of cannabis products. One or more user devices can provide user data to the hot box. The hot box can alert users of potential unsafe environmental conditions. The hot box can determine what is allowed by law to ensure legal compliance. The hot box can provide information to third parties. The hot box can further provide various amenities to the user.

### BRIEF DESCRIPTIONS OF THE DRAWINGS

FIG. 1 illustrates a system for providing cannabis to users discreetly for the user's personal use, according to various embodiments.

FIG. 2 illustrates a cannabis effect software, according to various embodiments.

FIG. 3 illustrates a sensor correlation database, according to various embodiments.

FIG. 4 illustrates an optical sensor software, according to various embodiments.

FIG. 5 illustrates an optical sensor database, according to various embodiments.

### DETAILED DESCRIPTION

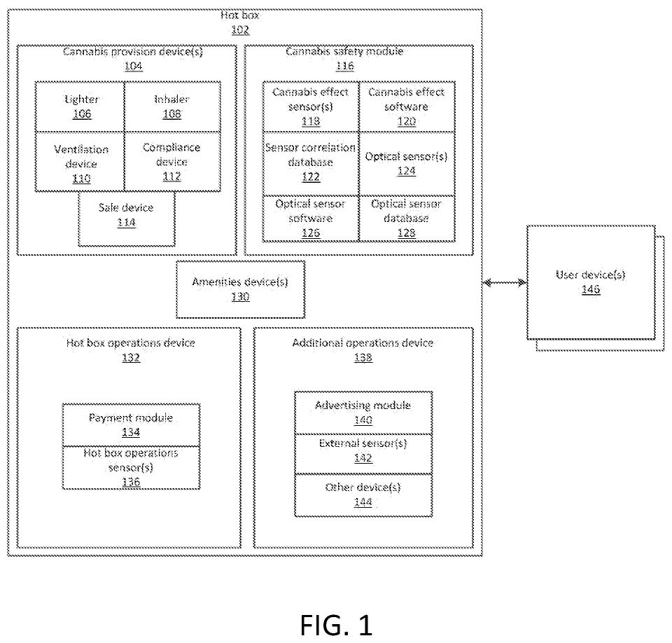

FIG. 1 illustrates a system for providing cannabis to users discreetly for the user's personal use, according to various embodiments. The system includes a discreet, standalone housing unit, referred to as a hot box 102. The hot box 102 is utilized for the discreet consumption of cannabis products by a user. In some embodiments, the hot box 102 may be a small construction (e.g., similar to a phone booth). In some embodiments, landowners can build the hot box 102 on their property and charge a fee for remote access. In other embodiments, the hot box 102 may be a portable, tent-like structure allowing the hot box 102 to be quickly constructed and provide a private location out of public view for cannabis consumption.

One or more cannabis provision device(s) 104 provides cannabis products to the user. A lighter 106 is a mechanism for igniting or otherwise combusting cannabis products. An inhaler 108, such as a vaping pen, is a mechanism for the user to inhale cannabis products. A ventilation device 110 is a mechanism for ventilating the interior of the hot box 102 and for, as necessary, filtering smoke and other consumption by-products from the air before it is exhausted to the exterior of the hot box 102. A legal compliance device 112 is a mechanism for monitoring the amount of cannabis provided to the user and providing only those amounts which are allowed by law. The legal compliance device 112 can consult a remote database maintained by a compliance organization, for example, to determine what is allowed by law. The legal compliance device 112 can include an age verification system. The legal compliance device 112 can be a vending machine. A sale device 114 is a mechanism for receiving payment for and providing cannabis to the user. The sale device 114 can be a vending machine.

A cannabis safety module 116 monitors the user's physiological effects stemming from cannabis use and matches the effects to potentially unsafe conditions. One or more cannabis effect sensor(s) 118 can sense data about the user, such as how much cannabis the user is consuming. The cannabis effect sensor 118 can also sense how much secondary smoke the user is getting, entering and exiting of the hot box, the user's movement, the user's pulse, etc. The cannabis effect sensor 118 can be an optical sensor. A cannabis effect software 120 matches the cannabis effect sensor data with medical data to alert the user of potential unsafe environmental conditions. A sensor correlation database 122 can include medical effects and conditions. One or more optical sensor(s) 124 are based on cameras or optical devices. Optical sensor software 126 is used with the optical cannabis sensor 124. An optical sensor database 128 includes optical sensor results and indicated effects.

One or more amenities device(s) 130 which provide amenities to the user during his or her cannabis consumption session. The amenities device 130 can provide the user with power supply, internet access, or food, drink, or water. The amenities may be complimentary. A hot box operations device 132 operates the hot box 102. A payment module 134 is a secure module that accepts payment from one or more users for the use of the cannabis. One or more hot box operations sensor(s) 136, such as a door sensor or a thermostat, are used in the operation of the hot box 102 as a paid service. An additional operations device 138 provides additional operations for the hot box 102 which are not related to cannabis consumption. The additional operations device 138 includes an advertising module 140 provides advertisements to the public around and outside the hot box 102. One or more external sensor(s) 142 sense the environment around and outside the hot box 102 and provide information to third parties, such as police or advertisers. One or more other devices 144 may be provided as part of the hot box 102, such as for a non-cannabis use that may promote discretion. One or more user device(s) 146 carried or worn by the user may provide data to the hot box 102 in the form of, for example, sensor data, medical data, or payment data.

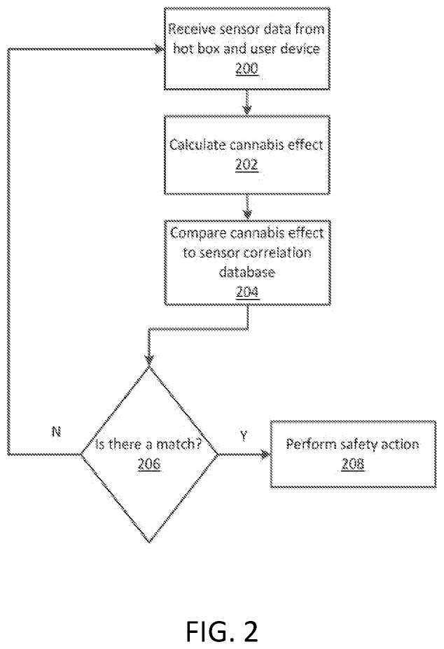

FIG. 2 illustrates a cannabis effect software, according to various embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process begins at 200 with the hot box 102 receiving cannabis effect sensor data from the cannabis effect sensor(s) 118 in the hot box 102 and receiving data from the user device 144. At 202, the effect of the cannabis upon the user is calculated based on the sensor data. At 204, the cannabis effect is compared to safety conditions in the sensor correlation database 122. At 206, it is determined if there is a match. If there is a match, then the process moves to 208 and the safety action correlated to the effect is performed. If there is not a match, then the process returns to the beginning at 200.

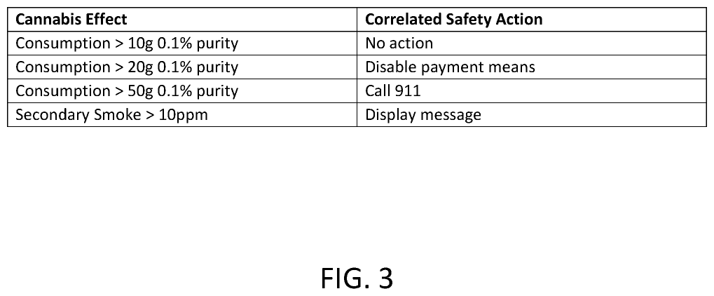

FIG. 3 illustrates a sensor correlation database, according to various embodiments One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The sensor correlation database 122 contains cannabis effects calculated from the sensor data and associated, correlated safety actions. For example, the correlated safety action for consumption greater than 10 g with 0.1% purity is no action at all. As another example, the correlated safety action for smoke greater than 10 parts per million (ppm) is to display a message to the user, such as “too smoky.”

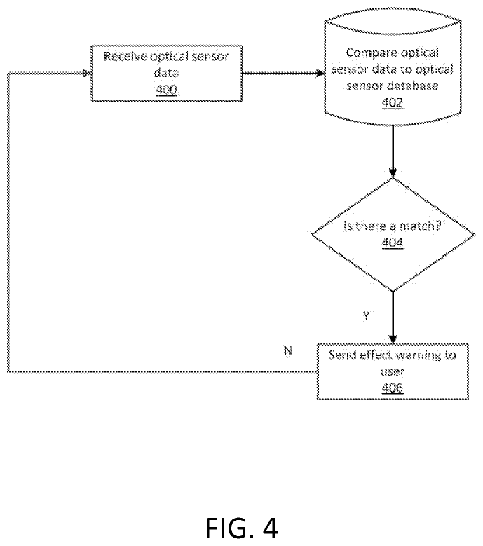

FIG. 4 illustrates an optical sensor software, according to various embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process begins at 400 when data is received from the one or more optical sensor(s) 124. At 402, the optical sensor data is compared to the optical sensor database 128. At 404, it is determined if there is a match between the optical sensor data and the optical sensor database 128. If there is a match, the process moves to 406 and sends a warning to the user of the effect. If there is not a match, the process returns to the beginning at 400.

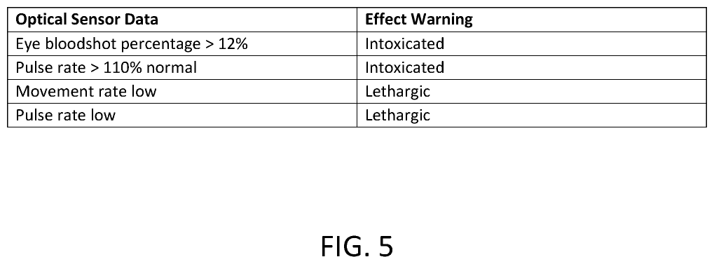

FIG. 5 illustrates an optical sensor database, according to various embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The optical sensor database 128 shows optical sensor data results and matches the optical sensor data results with indicated effect warnings, such as lethargic or intoxication. For example, if the optical sensor data result is that the users pulse rate is greater than 110% of a normal pulse rate then the indicated effect warning would be that the user is intoxicated. As another example, if the optical sensor data result is that the user's movement rate is low then the indicated effect warning would be that the user is lethargic.

What is claimed is:

1. A system for discreet consumption, the system comprising:
a hot box structure that encloses an internal space;
one or more cannabis provision devices located within the internal space enclosed by the hot box structure, each of the cannabis provision devices configured to dispense a type of cannabis product;
an operations controller device that controls access to the cannabis provision devices within the internal space based on received user data indicating eligibility of a user;
one or more sensors that monitor for one or more conditions within the internal space enclosed by the hot box structure; and
a database that stores one or more correlations between at least one of the conditions and a responsive action, wherein the responsive action includes displaying a message to the user.

2. The system of claim 1, wherein a selected one of the cannabis provision devices dispenses the respective type of cannabis product upon receipt of a payment by an associated sale device.

3. The system of claim 1, further comprising a compliance device that monitors a total amount of cannabis product dispensed to the user from the cannabis provision devices.

4. The system of claim 1, wherein the eligibility of the user is based on age, and further comprising a compliance device that verifies an age of the user based on the received user data.

5. The system of claim 4, wherein the compliance device verifies the age of the user by communicating over a communication network with a remote compliance database.

6. The system of claim 1, further comprising a ventilation device that ventilates the internal space enclosed by the hot box structure.

7. The system of claim 6, wherein the ventilation device filters smoke.

8. The system of claim 1, wherein the sensors include environmental sensors that monitor environmental conditions, the environmental sensors including a thermostat for measuring a temperature.

9. The system of claim 1, wherein the sensors include user sensors that monitor the user.

10. The system of claim 9, wherein the user sensors include an optical sensor for capturing image data regarding the user.

11. The system of claim 1, wherein each different one of the correlations is specific to a different level of the at least one condition and a different responsive action.

12. The system of claim 1, further comprising a safety module executable to identify when at least one of the conditions has been detected and to initiate the associated responsive action.

13. The system of claim 1, wherein the at least one condition is based on a total amount of the cannabis product dispensed to the user.

14. The system of claim 1, wherein the at least one condition is based on a total amount of the cannabis product identified as having been consumed by the user.

15. The system of claim 1, wherein the at least one condition is based on the type of the cannabis product dispensed to the user.

16. The system of claim 1, further comprising a communication interface, wherein the responsive action includes at least one of using the communication interface to display a warning message to the user within the enclosed internal space and send a warning message to a designated external recipient.

17. The system of claim 1, wherein at least one of the cannabis provision devices includes a lighter that is provided to the user upon dispensing the associated cannabis product.

18. The system of claim 1, wherein at least one of the cannabis provision devices includes an inhaler that is provided to the user upon dispensing the associated cannabis product.

19. The system of claim 1, further comprising one or more external sensors that capture data regarding the user prior to access, wherein the access into the internal space is further based on the captured data.
